(19) United States
(12) Patent Application Publication
(10) Pub. No.: US 2023/0346563 A1
(43) Pub. Date: Nov. 2, 2023

(54) Method for Designing a Mandibular Joint Prosthesis and Corresponding Production Method

(71) Applicant: Karl Leibinger Medizintechnik GmbH & Co. KG, Mühlheim/Donau (DE)

(72) Inventors: Adem Aksu, VS-Schwenningen (DE); Ralf Leibinger, Mühlheim/Donau (DE); Frank Reinauer, Emmingen-Liptingen (DE); Tobias Wolfram, Dreieich (DE)

(21) Appl. No.: 18/041,750

(22) PCT Filed: Jan. 17, 2022
(86) PCT No.: PCT/EP2022/050847

§ 371 Date: Feb. 15, 2023

(30) Foreign Application Priority Data
Feb. 11, 2021 (DE) 10 2021 201 278.7

(51) Int. Cl. A61F 2/30

(52) U.S. Cl. CPC A61F 2/30942; A61F 2/3099; A61F 2002/30461; A61F 2002/30578; A61F 2002/30948; A61F 2002/30991

(57) ABSTRACT

A method and a computer system is provided for designing a mandibular joint prosthesis for a patient's skull side to be treated. The method matches a standardized model of the mandibular joint to a graphic representation of the patient's healthy skull side and mirrors the model along a mirror plane onto a graphic representation of the skull side to be treated in order to make corresponding detailed adaptations.

The present invention relates to a method for designing an implantable mandibular joint prosthesis having a first implant part (mandible component) having an artificial joint head, which is fastenable on a mandible, and a second implant part (cranium component) having a joint surface, which is fastenable on a cranium, wherein the joint surface forms a buttress for the artificial joint head. In addition, the invention relates to a corresponding method for producing an implantable mandibular joint prosthesis.

The mandibular joint, which in medical language is also referred to as the TMJ (“temporomandibular joint”), is one of the most used and most important joints in the human body. It plays an essential role in the movement guidance of the mandible (the lower jaw), during chewing, speaking, swallowing, and stress management. It is even continuously in movement in sleep due to the swallowing movement.

Because of its diverse anatomy and its six degrees of freedom, the mandibular joint is also among the most complex joints of the human body. In order that the mandibular joint can carry out its movement sequences, the shape of the joint surfaces, the condition of the dentition, the tooth position, the tooth shape, and the chewing musculature have to form a functional system, which is subject to a certain susceptibility to malfunctions due to the large number of components. The left and right mandibular joint always work together here and are generally formed mirror symmetrical to one another.

However, if malfunctions arise in the area of the mandibular joint, for example functional restrictions or illnesses, significant negative effects on the everyday quality of life of patients can occur. Reestablishing correct joint structures is sometimes only possible in this case in the course of a surgical solution, i.e., by resection (i.e., removal) of the impaired joint and its replacement by a mandibular joint prosthesis.

A mandibular joint prosthesis is known, for example, from EP 3 003 225 B1, which provides a mechanism that permits a combined translation and rotation movement between skull and mandible, in which a sliding movement takes place between surfaces.

However, designing mandibular joint prostheses is a demanding task, since each prosthesis always only represents an incomplete approximation of the native structures and the prosthesis is typically designed on the drawing board or on the computer, without the functional interaction of the components to be articulated having been able to be checked in this case.

It is the object of the present invention to provide an improved method for designing a mandibular joint prosthesis, an improved production method for a mandibular joint prosthesis, and a computer system for designing a mandibular joint prosthesis.

The object is achieved by a method having the features of claim 1. A method is thus provided for designing a mandibular joint prosthesis for a skull side of a patient to be treated, having the following steps:
providing a three-dimensional, 3D, representation of the skull comprising at least the mandibular joints of the patient;
providing a three-dimensional, 3D, model for at least one functional group of the mandibular joint prosthesis, wherein the functional group comprises at least one condyle head and a fossa sliding surface for the condyle head;
arranging the provided 3D model with respect to the 3D representation of the skull on a reference skull side of the 3D representation, wherein the reference skull side is a skull side opposite to the skull side to be treated;
adapting the provided 3D model in size and alignment to the anatomical conditions of the 3D representation on the reference skull side;
mirroring a condyle head model from the reference skull side to the skull side to be treated, wherein the condyle head model is formed either by a condyle head of the 3D representation on the reference skull side or by at least one part of the condyle head of the 3D model;
extracting a fossa sliding surface from the adapted 3D model;
arranging the extracted fossa sliding surface on the skull side to be treated;
adapting the mirrored condyle head model and the fossa sliding surface to one another and/or to an anatomy of the 3D representation on the skull side to be treated;
outputting construction data for the mandibular joint prosthesis (in particular in the form of a mandibular joint prosthesis 3D model), which comprise at least the adapted mirrored condyle head model and the adapted fossa sliding surface.

In particular the fundamental concept of using structures on the healthy reference skull side in order to adapt a suitable 3D model and thereupon to carry out a mirroring onto the skull side to be treated enables as much information as possible from the healthy reference skull side to be taken into consideration for the design of the prosthesis. Not only good interaction of the implant parts of the mandibular joint prosthesis to be articulated with one another on the skull side to be treated can thus be achieved as such, but additionally also the most parallel and identical movement as possible on both skull sides.

Designing a mandibular joint prosthesis is to be understood in particular to mean planning or designing or laying out the mandibular joint prosthesis in such a way that construction data are provided, which ultimately only still have to be implemented by corresponding machines or solely mechanical actions to form the actual mandibular joint prosthesis.

Whenever reference is made herein to a “patient”, it is apparent that this means both female and also male patients.

The functional group of the mandibular joint prosthesis is to be understood as the group of elements or sections which articulate with one another, i.e., engage in one another, rub on one another, mutually support one another, exert counter forces on one another, and the like when the mandibular joint prosthesis is used instead of a natural mandibular joint. In other words, the functional group is to contain at least those elements which are designed to enable a movement of the mandibular joint by interaction with one another. In the mandibular joint prosthesis, these are thus at least the condyle head (or an element corresponding thereto) and a fossa sliding surface of the associated fossa articularis (or an element corresponding thereto).

The designation “skull” as used herein is to comprise in particular the cranium, the maxilla, and the mandible.

The terms “fossa” and “fossa articularis” are used synonymously herein. The terms “mandibular condyle”, “condyle”, and “condyle head” are also used synonymously.

Preferred refinements of the subjects according to the invention result from the dependent claims and the description, in particular with reference to the drawings.

According to several preferred embodiments, variants, or refinements of embodiments, the method comprises designing a fossa main body of the mandibular joint prosthesis in such a way that the fossa main body bears the fossa sliding surface. In this case it can be provided in particular that the fossa sliding surface of the later mandibular joint prosthesis is formed from a plastic, in particular a polyethylene, with which the fossa main body, which is preferably formed from titanium, is embodied as a material composite. For example, the fossa sliding surface can be bonded by means of a sintering process to the fossa main body.

Whenever reference is made herein to a formation of partial implants, sections, or elements from titanium, it is apparent that instead of titanium a suitable titanium alloy can also be used.

According to several preferred embodiments, variants, or refinements of embodiments, the adapted mirrored condyle head model is designed (or laid out) as part of a separate cranium component of the mandibular joint prosthesis and the adapted fossa sliding surface of the mandibular joint prosthesis is designed (or laid out) as part of a separate mandible component of the mandibular joint prosthesis. The cranium component and the mandible component can also be designated as separate partial implants (or implant parts). The formation of cranium component and mandible component as partial implants separate from one another first enables a great movement freedom of the two partial implants in relation to one another, which can expediently be restricted later by suitable means or measures, for example a suture, by a physician. Moreover, in this way the production is simplified, since the two partial implants can be produced separately from one another.

According to several preferred embodiments, variants, or refinements of embodiments, the cranium component and the mandible component are each designed in such a way that they comprise a respective eye (cranium component eye and mandible component eye), by means of which both the cranium component and the mandible component can be coupled to one another by a suture or a band. Such a suture can advantageously be made resorbable and can be fastened on the mandibular joint prosthesis inserted into the human body, in order to movably couple the cranium component and the mandible component to one another. This can help the patient in the initial familiarization phase to the mandibular joint prosthesis in learning the correct movements/articulations and positions of the mandibular joint prosthesis.

According to several preferred embodiments, variants, or refinements of embodiments, the method comprises a step of producing the mandibular joint prosthesis according to the output construction data, i.e., in particular according to the generated mandibular joint prosthesis 3D model. In this case, the method can also be referred to as a method for producing a mandibular joint prosthesis.

According to several preferred embodiments, variants, or refinements of embodiments, at least parts of the mandibular joint prosthesis are created by means of a generative (or additive) method using laser melting, in particular using titanium laser melting, so that the corresponding parts are formed completely or at least partially from titanium. Titanium is a material well suitable for mandibular joint prostheses, and high-quality, stable implants which are dimensionally accurate to the underlying 3D model at the same time may be produced by laser melting.

All parts or elements manufactured from titanium which articulate during the intended use of the mandibular joint prosthesis are preferably polished. Their coefficient of friction is thus once again reduced significantly, which enables even better articulation of the mandibular joint prosthesis. It is particularly preferred that the condyle head of the mandibular joint prosthesis (i.e., the element which fulfills those functions in the mandibular joint prosthesis which the condyle head fulfills in an intact mandibular joint) is polished.

According to several preferred embodiments, variants, or refinements of embodiments, the fossa sliding surface of the mandibular joint prosthesis is formed as a sliding layer made of a plastic, in particular from a polyethylene. The polyethylene can be in particular UHMWPE. UHMWPE is a thermoplastic polyethylene having ultra-high molecular weight, for example, of 2 to 8 million g/mol. Examples of UHMWPE are sold, for example, under the tradename “Celanese GUR® 1020”. The fossa sliding surface is preferably bonded with the aid of a sintering process to a fossa main body manufactured from titanium.

The combination of condyle head made of polished titanium and fossa sliding surface made of UHMWPE articulating with one another has proven to have particularly low friction and therefore to be particularly advantageous.

According to several preferred embodiments, variants, or refinements of embodiments, the mandible component has, in addition to a condyle head according to the condyle head model, at least one mandible fixation plate for fixing the mandible component on the mandible of the patient. This is typically provided with holes so that it can be fastened by means of screws on the mandible of the patient. However, other fastening means can also be provided and the mandible fixation plate can be designed accordingly. The mandible component is preferably manufactured in one piece from titanium, particularly preferably by titanium laser beam melting.

According to several preferred embodiments, variants, or refinements of embodiments, the cranium component has a cranium fixation plate for fixing the cranium component on the cranium of the patient. This is typically provided with holes so that it can be fastened by means of screws on the cranium of the patient. However, other fastening means can also be provided and the mandible fixation plate can be designed accordingly. The mandible component is preferably manufactured in one piece from titanium, particularly preferably by titanium laser beam melting.

The cranium component is preferably formed from a composite of titanium and a polyethylene, in particular UHMWPE.

According to several preferred embodiments, variants, or refinements of embodiments, in addition to the design and production of the mandibular joint prosthesis, the method also comprises a step of inserting or implanting the mandibular joint prosthesis in a patient. Optionally, a (preferably resorbable) suture can also be used for this purpose before, during, or after the insertion in order to couple the cranium component and the mandible component movably with one another through a respective cranium component eye and mandible component eye.

The invention additionally provides a computer system which is designed to carry out the method for creating the mandibular joint prosthesis.

The invention furthermore provides a computer program product which comprises executable program code which is designed, when it is executed, to carry out a method according to an embodiment of the method for designing the mandibular joint prosthesis.

The invention additionally also provides a computer-readable, nonvolatile data memory medium, which comprises executable program code, which is designed, when it is executed, to carry out a method according to an embodiment of the method for designing the mandibular joint prosthesis.

The invention additionally also provides a data stream, which comprises executable program code or is designed to provide such executable program code, wherein the executable program code is designed, when it is executed, to carry out a method according to an embodiment of the method for designing the mandibular joint prosthesis.

The invention additionally provides a web server, which is designed to provide or make accessible a method according to an embodiment of the method for designing the mandibular joint prosthesis as a web service.

The above embodiments and refinements may be combined with one another as desired, if reasonable. Further possible designs, refinements, and implementations of the invention also comprise combinations which are not explicitly mentioned of features of the invention described above or hereinafter with respect to the exemplary embodiments.

The invention is explained in more detail hereinafter on the basis of exemplary embodiments in the figures of the drawings. In the partially schematic figures:

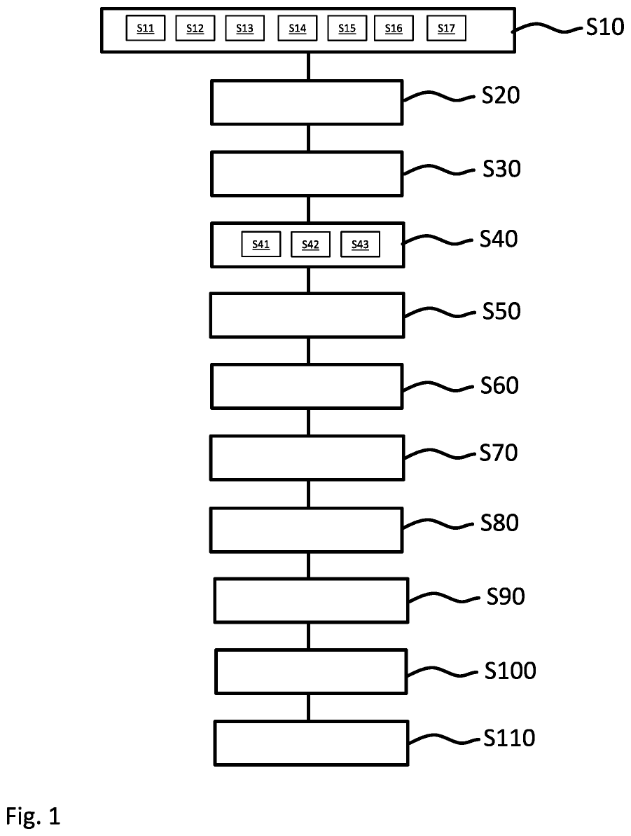

FIG. 1 shows a schematic flow chart to explain a method according to an embodiment of the present invention;

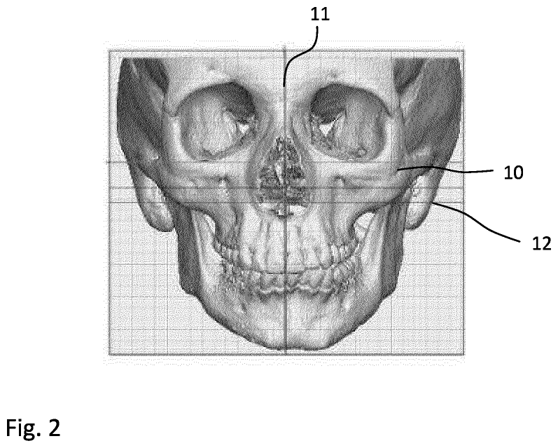

FIG. 2 shows a three-dimensional graphic representation of the skull of a patient;

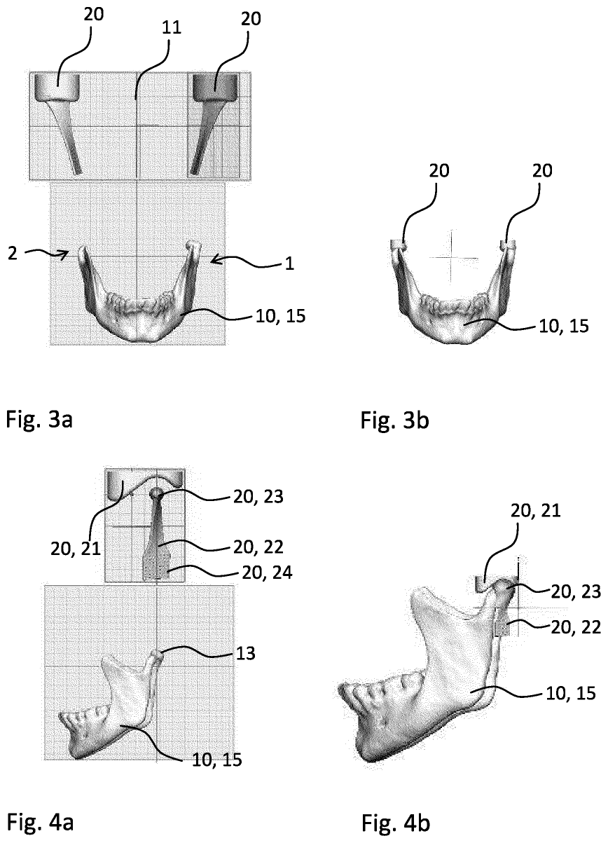

FIG. 3a shows a graphic representation of a mandible and a 3D model for a functional group of a mandibular joint prosthesis in a common graphic environment from the front;

FIG. 3b shows a graphic representation of the mandible and a 3D model for a functional group of a mandibular joint prosthesis in a common graphic environment from the front, after the 3D model has been adapted to the graphic representation of the mandible;

FIG. 4a shows a depiction of the situation from FIG. 3a from the side;

FIG. 4b shows a depiction of the situation from FIG. 3b from the side;

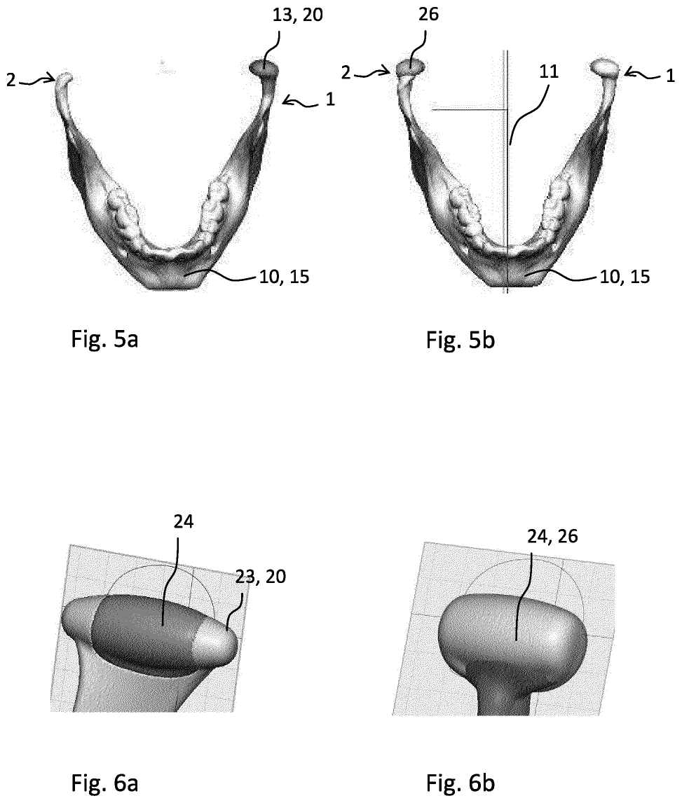

FIG. 5a shows a view from above of a graphic representation of a mandible having a condyle head marked on a healthy skull side and a defective condyle head on a skull side to be treated;

FIG. 5b shows the view from FIG. 5a, wherein a condyle head model was overlaid on the defective condyle head;

FIG. 6a shows a schematic detail view of the condyle head of the 3D model of the functional group of the mandibular joint prosthesis;

FIG. 6b shows a schematic detail view of a condyle head according to a condyle head model;

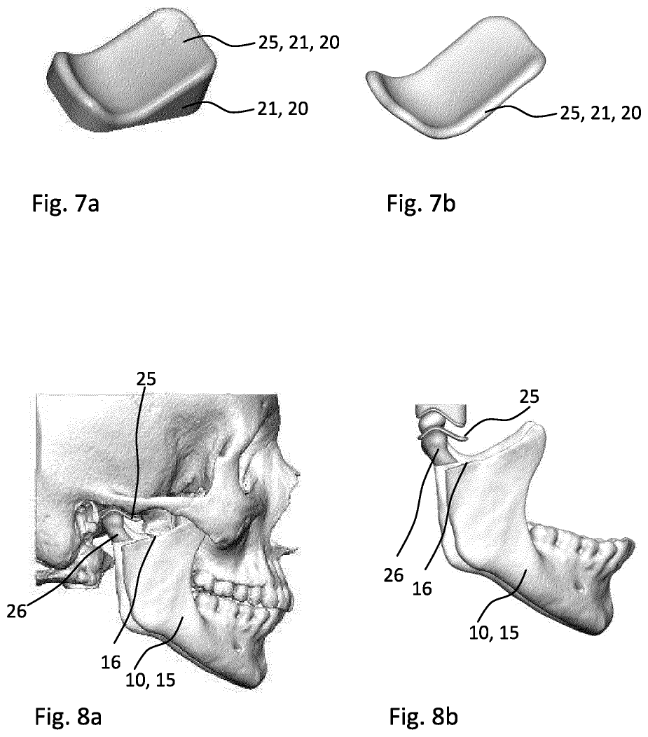

FIG. 7a shows a detail view of a fossa sliding surface of the 3D model of the functional group of the mandibular joint prosthesis;

FIG. 7b shows the fossa sliding surface from FIG. 7a after its extraction from the 3D model;

FIG. 8a shows a graphic representation of the skull of the patient after resection of the defective condyle head;

FIG. 8b shows the view from FIG. 8b, but with hidden cranium;

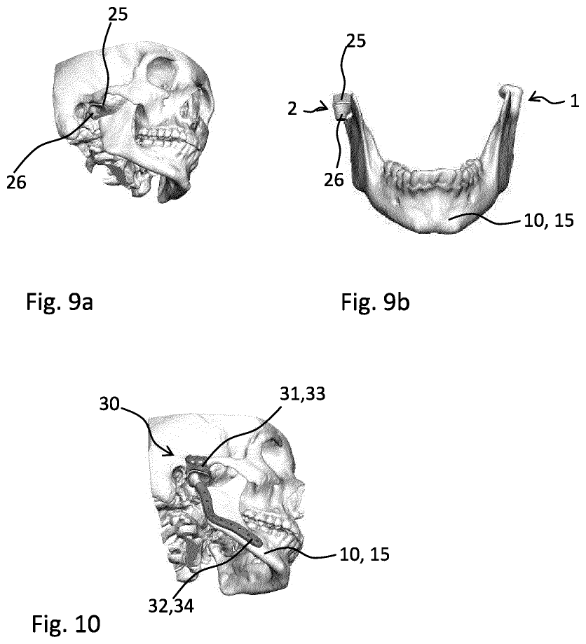

FIG. 9a shows a result of the adaptation of the functional group of a mandibular joint prosthesis 3D model to the skull anatomy of the patient diagonally from the bottom right;

FIG. 9a shows the situation from FIG. 9a, but viewed from the front with hidden cranium;

FIG. 10 shows a mandibular joint prosthesis 3D model arranged on the graphic representation of the skull of the patient;

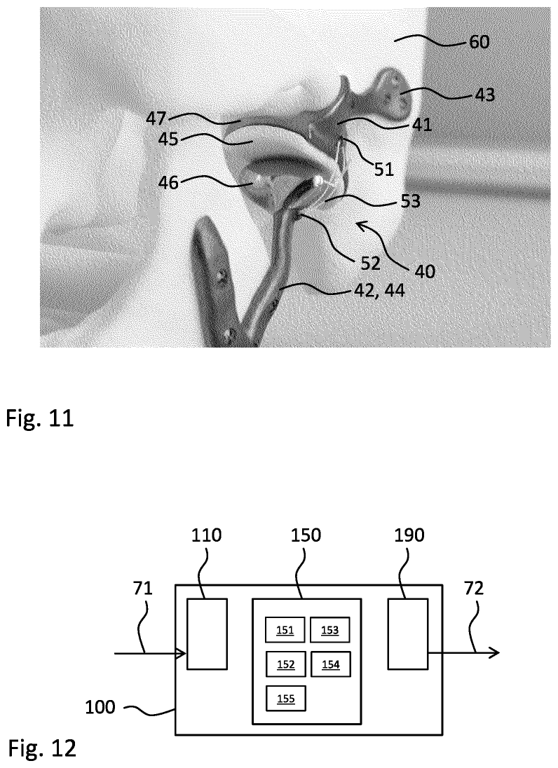

FIG. 11 shows a mandibular joint prosthesis, produced according to an embodiment of the present invention, attached to a skull model;

FIG. 12 shows a schematic representation of a computer system according to a further embodiment of the present invention;

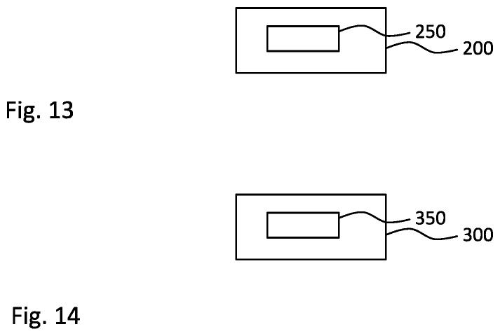

FIG. 13 shows a schematic representation of a computer program product according to a further embodiment of the present invention; and

FIG. 14 shows a schematic representation of a data memory medium according to a further embodiment of the present invention.

In all figures, identical or functionally identical elements and devices—if not indicated otherwise—have been provided with the same reference signs.

FIG. 1 shows a schematic flow chart to explain a method according to one embodiment of the present invention.

In a step S10, a three-dimensional (3D) representation of the skull of a patient is provided, wherein the representation comprises at least the mandibular joint (or possibly what remains thereof after an accident or illness).

For this purpose, initially in a step S11, anatomical data of the skull of the patient can be provided (read out, requested and received, etc.). The provision S11 of the anatomical data of the skull of the patient can take place, for example, using (or by provision of) medical imaging data, i.e., in particular data which have been generated by medical imaging methods, in particular tomography methods such as computer tomography (CT), digital volume tomography (DVT), or “cone beam computed tomography”, CBCT, magnetic resonance tomography (MRT), and the like.

Such medical imaging data are generally provided in the DICOM format, wherein other formats are also possible. “DICOM” is a registered trademark and stands for “Digital Imaging and Communications in Medicine”. Whenever reference is made herein to the DICOM standard, this can be understood in particular as the current version of the DICOM standard PS3.1 2020d, wherein structured reports according to a preceding or a subsequent DICOM standard version can also be used. The DICOM data can in particular be read out or requested from a PACS, wherein PACS stands for “Picture Archiving and Communication System”.

The imaging data are typically provided in the DICOM format as layered images, from which 3D data are generated by corresponding 3D image generation software. The 3D data can be provided, for example, in the STL format. The STL format describes the surface of 3D bodies with the aid of triangular facets, each of which is characterized by the three corner points and the associated surface normal of the triangle.

It is obvious that the original medical imaging data can have additional body parts and/or bones outside the skull of the patient. These can also be included in the provided anatomical data of the skull, or can be removed in an intermediate step, since the data about the anatomy of the bones of the skull are sufficient for the design and later production of the mandibular joint prosthesis.

In a step S12, segmenting can be carried out, i.e., in particular classification of pixels or voxels of the anatomical data as associated in each case with a specific bone (or other human body part), on the basis of the medical imaging data. For example, trained artificial neural networks are suitable for this purpose, in particular so-called “convolutional neural networks” (CNNs). Therefore, the parts of the human skeleton required for the present method, i.e., in particular the skull, can be recognized automatically on the basis of such segmenting and the relevant imaging data which relate to (i.e., comprise) these parts can optionally be isolated for further use.

The anatomical data can also be composed of medical imaging data from multiple sources. For example, dental scans (or “dental data”), for example, by way of conventional or 3D x-ray pictures of the teeth of the patient, can also be part of the 3D data. The dental scans can be based, for example, on classic plaster impressions of the teeth, which are either put through the same CT scanner as the patient was previously (the data are then provided as a DICOM image stack). Alternatively, dental scans can possibly be scanned using modern 3D oral scanners directly in the mouth of the patient. In the latter case, the dental scans are therefore advantageously provided at the same time in the STL format, and do not have to be generated via plaster impression/CT.

The relevant imaging data can subsequently, in a step S13, be read into 3D orthognathism software and processed therein together with the segmentation from step S12. The 3D orthognathism software can in turn generate 3D representations from the individual imaging data (for example, DICOM data such as DICOM layer images) and display them, for example, on a monitor, which is operatively connected to the computing device.

The provision S10 of the graphic representation can also comprise a preparation of the provided medical imaging data. If it should be necessary, for example, complete orthognathism planning can be carried out by means of the 3D orthognathism software, in order to define the position of maxilla and mandible exactly, and to depict a target occlusion, wherein dental data as described above can be used.

The term occlusion designates any contact of the teeth of the maxilla with those of the mandible. The planning or any other manipulation or preparation of the read-in imaging data or the generated 3D data can be carried out by means of an input device which is operatively connected to a computing device, which executes the software, and a display device (for example a monitor). The input device can comprise, for example, a keyboard, a computer mouse, a haptic input unit, or the like. The input device can also comprise a touchscreen, so that input device and monitor can also be integrated with one another, see also FIG. 12 and the associated description.

In a step S14, individual data structures or elements, in particular skull, maxilla, mandible, etc., can be exported from the orthognathism program together with a final splint, again in the STL format.

In a step S15, the various STL data from the 3D image generation software and/or the 3D orthognathism software can be read into a graphic user interface for modeling 3D structures (or 3D modeling software). Finally, in a step S16, a three-dimensional (3D) representation of the skull of the patient is generated in the graphic user interface, which is used as the basis for the further method.

Alternatively to steps S11-S16, which are used to provide S10 the 3D representation of the skull of the patient, a possibly already provided 3D representation of the skull can also be provided directly by reading out from a (volatile or nonvolatile) data memory. For example, an accurate 3D representation can already exist from a preceding intervention on the skull of the patient which took place shortly beforehand.

After the provision S10 of the anatomical data, a graphic representation of the skull is thus advantageously provided, at least in the area of the two mandibular joints, which comprises in particular the temporal bone and the mandible.

FIG. 2 schematically shows such a three-dimensional (3D) graphic representation 10 of the skull of the patient.

In a further step S17, a vertical mirror plane 11 (the sagittal plane) and/or a Frankfurt horizontal 12 can be introduced automatically into the graphic representation 10. If anatomical conditions in the 3D representation are not completely mirror symmetrical, the best matching sagittal plane can be automatically determined, for example, using an artificial intelligence entity such as a support vector machine. Such a mirror plane defined automatically or beforehand by a technician is preferably also checked and adapted if necessary by a physician.

In a step S20, a three-dimensional (3D) model for at least one functional group of the mandibular joint prosthesis, comprising at least one condyle head of the mandibular joint and a sliding surface for the condyle head of the mandibular joint, is provided, for example read in from 3D data sets. Such data sets can be provided, for example, in customary CAD software.

FIG. 3a and FIG. 4a each show such a model 20 in the upper area, wherein FIG. 3a shows a frontal view and FIG. 4a shows a side view. In the presently used example, the left side of the patient is the healthy skull side, while the right side is a skull side 2 to be treated. The healthy skull side is also referred to herein as the reference skull side 1, since it generally offers a reference for a functioning anatomical arrangement for the treatment. Only a graphic representation of the mandible 15 is shown in each case, wherein it is apparent in particular in FIG. 3a that on the skull side 2 to be treated, the condyle head of the mandible 15 has a defect. FIG. 4a shows the situation from FIG. 3a, wherein the view is of reference skull side 1.

A variety of models can be used for the model 20, which each have different characteristics with respect to complexity, functionality, and the like. It has surprisingly been shown that even relatively simple functional models are well suitable for this purpose. In this case, a few elementary geometric objects, such as torus sections, can be used to describe the functional group, divided into a mandible component 22 and a cranium component 21 functioning as a functional counter element. The mandible component 22 comprises the condyle head 23—preferably formed roughly in a spindle shape—of the 3D model 20. The cranium component 21 comprises fossa articularis and tuberculum articulare, each advantageously modeled from torus sections and connected to one another continuously and steadily.

For the specific design and dimensioning to scale of such a functional model, empirically determined mean values can be used in order to define the model 20. Once defined, the model 20 can thus be used as the foundation for all patients, as will be described in detail hereinafter.

In a step S30, the 3D model 20 is arranged with respect to the 3D representation 10 (i.e., in the same graphic representation or graphic environment, such as a virtual three-dimensional space), advantageously initially on the reference skull side 1. The graphic environment (which can also be referred to as a “graphic user interface”, GUI), can be displayed, for example, by means of a two-dimensional computer display screen. However, it is also conceivable that virtual reality or augmented reality devices are used, for example with the aid of virtual reality or augmented reality glasses. The three-dimensional character of a three-dimensional representation can often be represented objectively better with the aid of such devices, since the spatial imagination of a user is addressed or utilized directly.

In a step S40, the 3D model 20 is thereupon advantageously adapted in the graphic environment in such a way, i.e., in particular scaled S41, positioned S42, and/or oriented S43, that the 3D model 20 is in the intended functional alignment in relation to the graphic representation of the mandible 15. In other words, the 3D model 20 is arranged like the mandibular joint located on the reference side 1 is positioned and oriented, wherein care is to be taken in particular of the condyle head and the fossa. It can be formulated as a goal that the model contour of the 3D model 20 terminates nearly (i.e., essentially) flatly on both sides, i.e., both on cranium component 21 and mandible component 22. In other words, the 3D model 20 can be arranged so that the condyle head of the 3D model 20 could interact with the actual fossa on the reference skull side 1 (i.e., move and/or slide therein) and/or in such a way that the actual condyle head on the reference skull side 1 could interact with the fossa of the 3D model 20.

To adapt S40 the 3D model 20, the graphic user interface can have functions which enable a user to rotate, shift, rescale, etc. elements of the graphic representation 10 and/or elements (or the entirety) of the 3D model 20.

FIG. 3a and FIG. 4a each show the situation after step S30. In respective associated FIG. 3b or FIG. 4b, the situation is shown after carrying out step S40. It is particularly well visible in FIG. 4b how the condyle head 23 of the 3D model 20 is superimposed essentially congruent on the condyle head 13 (see FIG. 4a) on the reference skull side 1 of the graphic representation of the mandible 15.

In a step S50, a condyle head model is mirrored from the reference skull side to the skull side 2 to be treated at the mirror plane 11. FIG. 5a shows here in a view of the mandible 15 from above how the condyle head 13 of the 3D representation 10 is marked for use as the condyle head model 26 (see following description) on the reference skull side 1. FIG. 5b shows how, in the 3D representation 10 after step S50, the defective condyle head on the skull side 2 to be treated is overlaid with the condyle head model 26. By means of and in the graphic user environment, the user can also still perform post-corrections after step S50, in order to match the condyle head model 26 accurately to the anatomy of the skull side 2 to be treated, for example, to achieve soft transitions and flush termini.

The condyle head 13 of the reference skull side 1 of the 3D representation 10 is advantageously used as the condyle head model, if this condyle head 13 is free of defects. In this case, it can be presumed that this condyle head 13 can also be incorporated well into the anatomy of the patient on the skull side 2 to be treated. For this purpose, the condyle head 13, or a part thereof, of the reference skull side 1 of the 3D representation 10 can be marked for selection in the graphic environment by a user.

If this is not possible, for example, if the condyle head 13 on the reference skull side has a defect or has a shape which is not usable on the skull side 2 to be treated, another predefined 3D structure can also be used as the condyle head model.

For example, as schematically shown in FIG. 6a, at least a part of the condyle head 23 of the 3D model 20 can alternatively also be used as the condyle head model 26, such as a predetermined section 24 (or “detail” or “portion”), which has already been scaled beforehand according to step S40. FIG. 6b illustrates such a defined, rescaled, positioned, and oriented section 24, optionally after a rounding and/or detail adaptation to a section (for example a mandible fixation plate) of a mandible component of the mandibular joint prosthesis.

Furthermore, alternatively to the description above, another predetermined 3D structure can also be used as the condyle head model 26.

In a step S60, a surface geometry of the fossa is extracted from the 3D model 20, which represents the later fossa sliding surface of the mandibular joint prosthesis for the condyle head of the mandibular joint prosthesis, or is at least to be used as the base for it. In some variants, the 3D model 20 can already be conceived in such a way that the fossa is solely embodied therein as a 3D surface, i.e., solely consists of its fossa sliding surface 25.

FIG. 7a shows a section of the cranium component 21 of the 3D model 20 looking toward the fossa sliding surface 25. The indentation in the sliding surface 25 can be seen well, in which the condyle head (or its substitute) of the mandibular joint prosthesis is to engage later.

FIG. 7b shows the extracted fossa sliding surface 25 isolated.

In a step S70, the extracted fossa sliding surface 25 is arranged on the skull side to be treated. This can be carried out, as already described above with reference to the condyle head 23, by mirroring at the mirror plane 11. This has the advantage that due to the adaptation performed in step S40, the fossa sliding surface 25 on the reference skull side 1 is already aligned matching and it can therefore be presumed that after its mirroring at the mirror plane 11, an essentially matching alignment of the fossa sliding surface 25 on the skull side 2 to be treated also exists.

Alternatively, the fossa sliding surface 25 can also be extracted directly from the 3D model 20 rescaled in step S41 and thereupon arranged directly on the skull side 2 to be treated. Thereafter, in general further adaptation, positioning, and/or (re-)orientation steps can be necessary, similarly to above-described steps S41, S42, S43.

In a step S80, the mirrored condyle head model 26 and the fossa sliding surface 25 on the skull side 2 to be treated are adapted to one another and/or to an anatomy of the patient on the skull side 2 to be treated.

During a medical intervention, the damaged part of the mandibular joint of the patient can be removed (resection) on the skull side 2 to be treated. This and its result can already have been planned and taken into consideration in the graphic representation.

FIG. 8a shows the graphic representation 10 of the skull of the patient from the skull side 2 to be treated after completed removal of the graphic representation of defective condyle head 13. The graphic representation shown in FIG. 8a can either be the result of a simulated resection, i.e., an operation carried out solely on the graphic representation 10. Alternatively, the graphic representation 10 shown can also be based on medical imaging methods carried out after an actual resection, for example, on one or more tomography methods.

It is also very apparent in FIG. 8a how in step S80 the fossa sliding surface 25 and the condyle head model 26 can be adapted to one another and to the skull anatomy. The graphic user interface can enable various portions of the graphic representation 10 to be shown and hidden.

In FIG. 8b, for example, the case is shown in which the majority of the cranium has been hidden. Therefore, for example, after optimum adaptation (arrangement, orientation, etc.) of the fossa sliding surface 25 to the anatomy of the cranium, with hidden cranium, the arrangement, orientation, etc. of the condyle head model 26 can be finely adjusted to the fossa sliding surface 25 on the skull side 2 to be treated, in particular, as shown in FIG. 8b, with simultaneous observation, as a reference, of the interaction and arrangement of condyle head 13 and associated fossa sliding surface on the reference skull side 1. In addition, the resection boundary 16 is also visible in FIG. 8a and FIG. 8b, which, as already mentioned, can represent either an actual resection boundary or a virtual, solely planned or estimated resection boundary.

FIG. 9a and FIG. 9b show a final result of the adaptation in step S80 from two different perspectives: FIG. 9a shows the functional group of the planned mandibular joint prosthesis, namely the fossa sliding surface 25 and the condyle head model 26, diagonally from below with cranium largely shown. FIG. 9b shows the mandible 15 with added fossa sliding surface 25 and added condyle head model 26 directly from the front, wherein the majority of the cranium is hidden, and the view represents the functional group of the planned mandibular joint prosthesis together with the functional group of the mandibular joint on the healthy reference skull side 1.

Both representations, according to FIG. 9a and FIG. 9b, can be rotated, enlarged, shrunk, and changed in the display in other ways as desired by the user in the graphic user interface, so that the user can achieve an optimum adaptation to the anatomical conditions of the patient.

In a step S90, based on the functional group made up of condyle head model 26 and fossa sliding surface 26 adapted in step S80, a mandibular joint prosthesis 3D model is generated. For this purpose, in particular transitions to the native bone situation can be designed specifically for the patient and laid out based on a design guideline (for example having a defined minimum dimensioning).

FIG. 10 shows by way of example such a generated mandibular joint prosthesis 3D model 30, arranged on the graphic representation 10 of the skull of the patient precisely as in reality the mandibular joint prosthesis itself is later to be attached to the actual skull of the patient.

The mandibular joint prosthesis 3D model 30 comprises in this case a cranium component 31 and a mandible component 32. The cranium component 31 comprises the fossa sliding surface 25 and a cranium fixation plate 33 connected thereto. The mandible component 32 comprises the one condyle head according to the condyle head model 26 and a mandible fixation plate 34. In FIG. 10, the mandible fixation plate 34 is shown as an elongated, deformed strip. Depending on the specific anatomical conditions of the patient, however, the mandible fixation plate 34 can also have other shapes. For example, the mandible fixation plate 34 can also have one or more transverse offshoots in addition to its elongated shape along the outer contour of the mandible and/or can be angled.

The cranium component 31 can be designed so that it has a fossa main body, which bears the fossa sliding surface 26, wherein it is the fossa main body on which the cranium fixation plate 33 is fastened or issues from.

The cranium fixation plate 33 preferably has openings into which fastening means, for example screws, can be introduced to fasten the cranium fixation plate 33 on the cranium of the patient. The mandible fixation plate 34 preferably has openings in which fastening means, for example screws, can be introduced to fasten the mandible fixation plate 34 on the mandible of the patient.

Optionally, an eye can be provided in each case on the cranium component 31 and on the mandible component 32. A suture or a band can be guided through these two eyes and knotted, so that the then closed suture (or the like) movably couples the two eyes and thus the cranium component 31 and the mandible component 32 to one another, see also FIG. 11 and the associated description in this regard.

The designing of the mandibular joint prosthesis 3D model 30 in such a way that it comprises the mentioned eyes advantageously takes place as the last step in generating the mandibular joint prosthesis 3D model 30, since at this point in time the movements and degrees of freedom to be expected, which the mandibular joint prosthesis will permit, are apparent.

In a step S100, construction data can be generated and output based on the generated mandibular joint prosthesis 3D model 30. For example, the construction data can be output entirely or partially to one or more machines for automatic production of a mandibular joint prosthesis 40 according to the mandibular joint prosthesis 3D model 30. Together with the construction data, control signals can also be output, which control the machines accordingly.

In an optional step S110, the mandibular joint prosthesis can be produced according to the output construction data, i.e., according to the mandibular joint prosthesis 3D model 30, as explained hereinafter. In embodiments in which the method comprises this step S110, the method can also be referred to as a method for producing a mandibular joint prosthesis. Of course, step S110 for producing the mandibular joint prosthesis based on the construction data can also be carried out independently of the preceding steps, in particular separated spatially and/or chronologically from the execution of the preceding steps.

In an optional further step, the mandibular joint prosthesis can finally be used in a patient.

FIG. 11 shows an exemplary illustration, for which a mandibular joint prosthesis 40, which was laid out and produced according to the present invention, was fastened for illustration to a skull model 60.

FIG. 11 shows here how the mandibular joint prosthesis 40 (very similarly to the underlying mandibular joint prosthesis 3D model 30) has a cranium component 41 and a mandible component 42.

The cranium component 41 comprises a cranium fixation plate 43, which was screwed using screws onto the skull model 60 in the situation shown in FIG. 11. The cranium component 41 additionally comprises a fossa main body 47, which is connected to the cranium fixation plate 43 and is designed to bear the fossa sliding surface 45. Cranium fixation plate 43 and fossa main body 47 can advantageously be formed in one piece, in particular generatively (or, in other words additively), preferably in a method for selective laser melting of titanium.

The fossa sliding surface 45, in contrast, is advantageously formed from a polyethylene material, preferably from “ultra-high-molecular-weight polyethylene” (abbreviated by “UHMWPE” or sometimes also by “UHMW”). UHMWPE is a thermoplastic polyethylene having ultra-high molecular weight, for example of 2 to 8 million g/mol. Preferably, the fossa sliding surface 45 is bonded with the aid of a sintering process to the fossa main body manufactured from titanium. The cranium component 41 as a whole can thus be formed from a composite material made of titanium and UHMWPE. The component formed from UHMWPE can be melted, for example, as an independent component, having only one face as an adhesive base. A wall in the style of an inlay surrounding the UHMWPE component is thus possible, but is not required.

The mandible component 42 comprises, in addition to the condyle head 46, which is formed precisely corresponding to the condyle head model 26 in the mandibular joint prosthesis 3D model 30, the mandible fixation plate 44. In the example shown in FIG. 11, the mandible fixation plate has an angled section, which extends in the direction of the crown extension of the mandible.

The mandible component 42 is preferably produced in one piece, in particular generatively (or, in other words additively), particularly preferably in a method for selective laser melting of titanium. To improve the sliding capability of the condyle head 46 on the fossa sliding surface 45, i.e., to make the articulation pairing have as low friction as possible, the condyle head 46 is advantageously polished. The sliding pair between the fundamentally separate partial implants of the cranium component 41 and the mandible component 42 thus preferably consists of UHMWPE and polished titanium.

However, it is also conceivable to produce the mandible component 42 likewise as a composite component, wherein in particular the condyle head 46 can be formed from a different material than the remainder of the mandible component 42, in particular the mandible fixation plate 44. For example, the condyle head 46 can be formed from a ceramic material.

FIG. 11 additionally also shows a cranium component eye 51 attached to the cranium component 41 and a mandible component eye 52 attached to the mandible component 42. In addition, a ring-shaped closed suture 53 coupling the cranium component eye 51 and the mandible component eye 52 is shown.

The suture 53 can be attached at the eyes 51, 52 before or after the use of the mandibular joint prosthesis 40 on the patient. Due to the complex movement functions of the mandibular joint and the sometimes slightly different functions of the mandibular joint prosthesis 40, the suture 53 can initially offer the patient assistance until the brain of the patient has processed the new situation. In particular, the suture 53 can prevent the patient from moving cranium component 41 and mandible component 42 too far away from one another, i.e., the suture 53 can fulfill a holding function or, in other words, can permit reference movements.

The suture 53 (or the band or the like) can advantageously be formed from a resorbable material. The resorption duration of the material is advantageously dimensioned in such a way that the suture 53 is completely resorbed at a point in time at which in the majority of the cases its function is no longer necessary. This is specifically when the healing process is advanced and the new positions and movement sequences are envisioned for the patient and appear natural, maxilla and mandible are generally again held and controlled by the natural muscle loops, as is also the case in the native/healthy situation. This is incorporated into the overall concept behind the present invention, according to which the mandibular joint prosthesis 40 is to be as close as possible to the natural anatomic conditions and functionalities of not only an average patient, but very specifically to each individual patient.

FIG. 12 shows a schematic representation of a computer system 100 according to another embodiment of the present invention. The computer system 100 is designed and configured to carry out the method according to the invention for designing a mandibular joint prosthesis 40, preferably the method described above with reference to FIGS. 1 to 11. In particular, the computer system 100 can consist for this purpose of a personal computer, PC, on which corresponding software is executed. The software can be designed to enable the user to carry out steps S10 to S100 described above.

The computer system 100 can comprise an input interface 110, by means of which medical data 71 of a patient can be read in. The medical data 71 can be medical image data, processed data, and/or the like, as was described above in particular with reference to step S10. In particular, the medical data 71 can comprise DICOM data, such as tomography image data, dental data, and the like. The medical data 71 can be received and/or requested for example from a PACS (“picture archiving and communication system”), from a cloud storage solution, or the like.

From the input interface 110, the received medical data 71 are transmitted to a computing device 150, which typically has a central processor unit 151 (CPU), a working memory 152 (“random access memory”, RAM), a nonvolatile data memory 153, a display device 154 (e.g., monitor, AR or VR glasses, or touchscreen), and a user input interface 155 (e.g., keyboard and computer mouse, touchscreen, etc.), which are operatively coupled to one another to execute the software and carry out the method. The software can be stored for this purpose in the nonvolatile data memory 153 and can be executed by the central processor unit 151 in the working memory 152. Alternatively or additionally, the software, or individual modules or plug-ins of the software, can also be provided as a web service.

The computer system 100 additionally comprises an output interface 190, which is designed to output the construction data 72 generated by the computing device 150 in the method in step S100, for example, to a data buffer memory, to a machine for producing one or more parts of the mandibular joint prosthesis 40, for example, a machine for creating implant parts using laser melting, or the like.

FIG. 13 shows a schematic representation of a computer program product 200, which comprises executable program code 250, which is designed, when it is executed, to carry out a method according to an embodiment of the method for designing a mandibular joint prosthesis 40, in particular the method which was described above on the basis of FIGS. 1 to 11.

FIG. 14 shows a schematic representation of a nonvolatile, computer-readable data memory medium 300, which comprises executable program code 350 which is designed, when it is executed, to carry out a method according to an embodiment of the method for designing a mandibular joint prosthesis 40, in particular the method which was described above on the basis of FIGS. 1 to 11.

Although the present invention was described above on the basis of preferred exemplary embodiments, it is not restricted thereto, but is modifiable in a variety of ways. In particular, the invention may be changed or modified in manifold ways without deviating from the core concept of the invention.

In summary, the invention provides a method and a computer system for designing a mandibular joint prosthesis for a skull side of a patient to be treated. A core concept of the method is to align a standardized model of the mandibular joint to a graphic representation of the healthy skull side of the patient, and then to mirror this at a mirror plane to form a graphic representation of the skull side to be treated, in order to also perform detailed adaptations to the model there.

### LIST OF REFERENCE SIGNS

1 reference skull side
2 skull side to be treated
10 graphic representation of the skull
11 mirror plane
12 Frankfurt horizontal
13 condyle head of the graphic representation of the mandible
graphic representation of the mandible
16 resection boundary
20 3D model
21 cranium component of the 3D model
22 mandible component of the 3D model
23 condyle head of the 3D model
24 section of the condyle head of the 3D model
25 sliding surface of the fossa of the 3D model
26 condyle head model
30 mandibular joint prosthesis 3D model
31 cranium component of the mandibular joint prosthesis 3D model
32 mandible component of the mandibular joint prosthesis 3D model
33 cranium fixation plate of the mandibular joint prosthesis 3D model
34 mandible fixation plate of the mandibular joint prosthesis 3D model
40 mandibular joint prosthesis
41 cranium component of the mandibular joint prosthesis
42 mandible component of the mandibular joint prosthesis
43 cranium fixation plate of the mandibular joint prosthesis
44 mandible fixation plate of the mandibular joint prosthesis
45 fossa sliding surface of the mandibular joint prosthesis
46 condyle head of the mandibular joint prosthesis
47 fossa main body of the mandibular joint prosthesis
51 cranium component eye
52 mandible component eye
53 suture
60 skull model
71 medical data
72 construction data
100 computer system
110 input interface
150 computing device
151 central processor unit
152 working memory
153 nonvolatile data memory
154 display device
155 user input interface
190 output interface
S10 . . . S110 method steps

1. A method for designing a mandibular joint prosthesis for a skull side of a patient to be treated, having the following steps:
providing a three-dimensional (3D) representation of the skull comprising at least the mandibular joint of the patient;
providing a three-dimensional, (3D) model for at least one functional group of the mandibular joint prosthesis, wherein the functional group includes at least one condyle head and a fossa sliding surface for the condyle head;
arranging the provided 3D model with respect to the 3D representation of the skull on a reference skull side of the 3D representation, wherein the reference skull side is a skull side opposite to the skull side to be treated;
adapting the provided 3D model in size and alignment to the anatomical conditions of the 3D representation on the reference skull side;
mirroring a condyle head model from the reference skull side on the skull side to be treated, wherein the condyle head model is formed either by a condyle head of the 3D representation on the reference skull side or by at least one part of the condyle head of the 3D model;
extracting a fossa sliding surface from the adapted 3D model;
arranging the extracted fossa sliding surface on the skull side to be treated;
adapting the mirrored condyle head model and the fossa sliding surface to one another and/or to an anatomy of the 3D representation on the skull side to be treated; and
outputting construction data for the mandibular joint prosthesis, which include at least the adapted mirrored condyle head model (26) and the adapted fossa sliding surface.

2. The method as claimed in claim 1, further comprising:
designing a fossa main body of the mandibular joint prosthesis in such a way that the fossa main body bears the fossa sliding surface.

3. The method as claimed in claim 1, wherein:
the adapted mirrored condyle head model is designed as part of a separate cranium component of the mandibular joint prosthesis; and
the adapted fossa sliding surface of the mandibular joint prosthesis is designed as part of a separate mandible component of the mandibular joint prosthesis.

4. The method as claimed in claim 3, wherein the cranium component and the mandible component are each designed in such a way that they include a respective eye, by means of which the cranium component and the mandible component can be coupled to one another by a suture or a band.

5. The method as claimed in claim 1, further comprising:
producing the mandibular joint prosthesis according to the output construction data.

6. The method as claimed in claim 5, wherein at least parts of the mandibular joint prosthesis are created by means of a generative method using laser melting.

7. The method as claimed in claim 5, wherein the fossa sliding surface of the mandibular joint prosthesis is formed as a sliding layer made of a plastic, in particular made of a polyethylene.

8. The method as claimed in claim 5, wherein the mandible component, in addition to a condyle head according to the condyle head model, has at least one mandible fixation plate for fixing the mandible component on the mandible of the patient; and wherein the mandible component is manufactured in one piece from titanium.

9. The method as claimed in claim 5, wherein the cranium component is formed from a composite of titanium and a polyethylene and has a cranium fixation plate for fixing the cranium component on the cranium of the patient, which cranium fixation plate is formed from titanium.

10. A computer system, which is configured to execute program code to carry out the method as claimed in claim 1.
